(19) United States
(12) Patent Application Publication
(10) Pub. No.: US 2011/0125110 A1
(43) Pub. Date: May 26, 2011

(54) MATERIAL FOR USE AS A WOUND PACKING ELEMENT, PARTICULARLY IN NEGATIVE PRESSURE WOUND THERAPY

(75) Inventor: Stephen Cotton, Nottinghamshire (GB)

(73) Assignee: BRIGHTWAKE LIMITED, Nottingham, Nottinghamshire (GB)

(21) Appl. No.: 13/055,676

(22) PCT Filed: Jul. 23, 2009
(86) PCT No.: PCT/GB2009/050910

§ 371 Date: Jan. 24, 2011

(30) Foreign Application Priority Data
Jul. 24, 2008 (GB) 0813542.8

(51) Int. Cl. A61M 1/00

(52) U.S. Cl. 604/290

(57) ABSTRACT

A wound packing element is in the form of a nonwoven material, particularly an airlaid nonwoven material. Such a wound packing element, or a number of such wound packing elements formed into a layered assembly, may be utilised to pack a wound cavity. The wound packing element is of particular utility in negative pressure wound therapy (NPWT).

This invention relates generally to wound care. More specifically, this invention relates to wound packing elements for packing wound cavities, particularly during negative pressure wound therapy.

Negative pressure wound therapy (NPWT) involves the application of a pressure that is reduced relative to that of the surroundings (commonly referred to as “negative pressure”) to a wound, which causes mechanical contraction of the wound and removal of wound fluid, thus promoting formation of granulation tissue and accelerating wound closure. The technique is particularly effective in the treatment of slow healing wounds such as chronic leg ulcers and large open wounds. A dressing consisting of an occlusive drape, traversed by a drainage tube, is applied to the wound opening, forming a seal under which a negative pressure can operate. The drainage tube is connected to a negative pressure source allowing the wound fluid to be drawn away. In the case of large open wounds, the wound cavity must be packed with a wound packing element to prevent the dressing from being drawn into the wound cavity by suction and to ensure an even distribution of pressure throughout the wound.

A wound packing element must effectively fill a wound cavity, contacting the entire surface of the wound with substantially even pressure. The material must be sufficiently compactable to enable contraction with the wound cavity when a negative pressure is applied, while also being firm enough to prevent the dressing from being drawn into the wound. The packing material must permit free passage of fluid without becoming clogged to ensure an even distribution of pressure within the wound cavity, and preferably be non-adherent to the wound surface. Currently, wound packing elements consist of either gauzes or foams.

Gauze is typically applied as a single layer, a drain is placed on the gauze and then a second piece of gauze is placed over the drain, creating a “gauze-sandwich”. Gauze is most suitable as a packing element for smaller wounds and has a tendency to fragment upon redressing, which could potentially result in fibres remaining in the wound.

Foams are generally used for packing large wound cavities. A range of foams with different properties are available, such as polyurethane foam (black) and polyvinylalcohol (PVA) (white) foam. PVA foam is denser and less permeable than polyurethane and requires a higher negative pressure to function effectively. The choice of foam depends on the application; for example, the more porous polyurethane foam is more commonly used on larger or deeper wounds. A combination of polyurethane and PVA foam can be used, depending on the desired result. Foam can be cut to fit the size and shape of the wound, and multiple pieces of foam may be used if necessary, although each piece of foam must come into contact with another piece of foam in order to achieve uniform compression when a negative pressure is applied. However, foams with sufficient density to effectively pack a wound often lack the required permeability and are often subject to clogging. Also, the process by which foam is manufactured is poorly controlled, which can potentially lead to the introduction of unwanted agents into the material.

The current choice of materials for use as wound packing elements, particularly in NPWT, is limited, and those that are available are not ideal for the purpose.

This invention provides a new form of wound packing element, which overcomes or substantially mitigates the above-mentioned and/or other limitations of the prior art. The invention further provides methods of treatment of wounds that utilise the novel form of wound packing element.

In the first aspect of the invention, there is provided a wound packing element in the form of nonwoven material.

The wound packing element according to the invention is advantageous primarily because of the physical properties of the material. Where generally elastic inter-fibre bonds are present, nonwoven material may be firm but highly pliable and elastic. The material may be readily deformable but resilient. An open fibre structure means a considerable volume of the material consists of air spaces, imparting both high permeability and substantial compactability to the material. Consequently, the thickness of the material may be reduced substantially by the application of a mechanical force, upon removal of which the material returns to its original dimensions.

It is essential that a material used as a wound packing element is firm enough to provide sufficient resistance to prevent the dressing from being sucked into the wound cavity. Foam products of such firmness generally have a relatively high density and thus a reduced permeability, increasing their tendency to clog. Clogging of a wound packing element may cause the uneven distribution of pressure throughout a wound cavity by obstructing fluid transfer, but at the very least will reduce the rate of fluid removal. Where the fibre structure is open, nonwoven material is able to combine sufficient firmness with high permeability.

In a related aspect of the invention, there is provided a method of packing a wound cavity, which method comprises the insertion into the wound cavity of one or more wound packing elements in the form of nonwoven material.

Two or more wound packing elements of the invention may be used in combination to pack a wound. Typically, such wound packing elements have the form of sheets that may be formed into a stack to fill the wound cavity. Thus, in a second aspect of the invention, there is provided a wound packing comprising a layered assembly of two or more wound packing elements, wherein the wound packing elements are in the form of nonwoven material.

In yet a further aspect of the invention, there is provided a method for packing a wound cavity, which comprises forming a layered assembly of wound packing elements, wherein some or all of the wound packing elements are in the form of nonwoven material, and positioning the assembly so formed within the wound cavity.

The methods of the invention are particularly useful for packing a wound cavity in negative pressure wound therapy. Thus, in a further aspect of the invention, there is provided a method of negative pressure wound therapy, which method comprises packing a wound cavity with one or more wound packing elements in the form of nonwoven material, and applying reduced pressure to the wound cavity.

Wound packing elements according to the first aspect of the invention typically have the form of planar sheets of material. Such sheets typically have thicknesses of from about 5 mm to several tens of millimetres, eg about 100 mm. The thickness of the wound packing element will commonly be greater than 5 mm, or greater than 10 mm. The wound packing element will typically have a thickness that is less than 50 mm, eg less than 40 mm or less than 30 mm. Typically, the thickness of the wound packing element may be in the range 5 mm to 50 mm, more typically 10 mm to 50 mm.

Wound packing elements according to the invention may be manufactured in standard sizes and shapes to suit differing sizes and shapes of wound. Thus, the wound packing elements may be produced in shapes that are generally square, rectangular, circular or ovoid, and in sizes in which the major dimensions are from a few centimetres to several tens of centimetres or more. Alternatively, the wound packing elements may be supplied in the form of oversized sheets which may be cut to the appropriate dimensions immediately prior to use, to fit a wound cavity. In large wounds, wound packing elements may also be layered upon each other or rolled, folded, or otherwise deformed in order to fill the wound cavity as desired.

Wound packing elements according to the invention are manufactured using nonwoven materials. Various methods for the production of nonwoven materials will be familiar to those skilled in the art. However, preferred materials for use in the invention are produced by air-laying. Airlaid nonwoven materials are therefore a preferred class of nonwoven materials for use in the invention.

Airlaid nonwoven materials are currently utilised in a range of applications including filtration and cushioning, eg in mattresses. Airlaid nonwoven material is also used in a range or personal care products, where it is the absorbency of the material that is utilised. The use of airlaid nonwoven material as a wound packing element, particularly for use in NPWT, has not previously been disclosed.

Airlaid nonwoven material is manufactured by dispersing fibres into a fast moving air stream and condensing them in progressive layers onto a screen using either pressure or a vacuum, to produce an airlaid web. These fibres are then bonded either by heat (thermal bonding), involving heating an airlaid web composed of synthetic fibres to the point where the fibres fuse together, or using a synthetic binding substance (latex bonding). A combination of these methods may also be used (multi-bonding), where an airlaid material is produced by thermal bonding, followed by spraying with a synthetic binder to reduce lint release. The process of producing airlaid nonwoven material is highly controllable and it is possible to incorporate a range of different fibres, or fibre densities, into a single layer of material. Consequently, the manufacturing process can be tailored to provide wound packing materials with a variety of properties, so the porosity and firmness may be varied depending on the particular application.

For use in the present invention, in the manufacture of wound packing elements, it is most desirable to use thermally bonded airlaid nonwoven material because the process is cleaner than the alternatives that involve the introduction of a binding agent into the material. The use of a combination of thermal and latex bonding may, however, also be desirable to reduce contamination of the wound with fibre fragments.

Airlaid nonwoven material is generally highly durable, retaining its properties over time in a way that compares favourably with other materials such as foam. Consequently, the shelf life of any product composed of airlaid material will compare favourably with that of other materials.

The wound packing elements of the invention may be manufactured from fibres of a wide range of materials. Most preferably, the material is a synthetic polymeric material. A wide range of synthetic polymeric materials may be employed, including polyesters, polyacrylics, polyamides, polyolefins and polylactides, amongst many others.

A particularly preferred material for use in the invention is polyester. Polyester is generally biologically inert, with the result that adverse reactions to it are unlikely.

One suitable airlaid and thermally bonded nonwoven polyester material for use in the invention is that sold under the trade name AEROFILL by Libeltex BVBA (Marialoopsteenweg 51, BE-8760 Meulebeke, Belgium), particularly the 900 gsm grade of that material.

Ideally, a wound packing element should be non-adherent to prevent attachment to the surface of the wound while in place, causing difficulties during redressing as well as pain and trauma for the patient. Because of the nature of a healing wound, cellular infiltration into a porous material such as a wound packing element is a possibility. In order to reduce the likelihood of this occurring, the wound packing element can be coated with a porous, non-adherent layer, which would prevent infiltration of progressively healing tissue into the packing element or sticking of the packing element to the wound surface by another mechanism.

The nonwoven material may be coated or impregnated with one or more agents that complement its function as a wound packing element. These may include agents to enhance the healing process, painkillers or particularly antimicrobials. An antimicrobial agent such as silver can be incorporated into the fibres from which the nonwoven material is manufactured. Other antimicrobial agents such as honey or triclosan can be incorporated into the material after manufacture.

The wound packing elements of the invention are preferably supplied pre-sterilised in sealed packaging. Where the nonwoven material is sensitive to heat, sterilisation methods using heat and pressure are not suitable. More preferred methods include chemical sterilisation using an agent such as ethylene oxide, which is commonly used for sterilising other medical equipment, or gamma irradiation.

Because the material does not necessarily have a specific secondary structure, it can be provided in large sheets which can then be conveniently cut to the appropriate dimensions and used as a wound packing element in whichever way is most desirable. The material is pliable enough to be rolled, folded, or otherwise deformed, to fit into a wound cavity in whatever way is most suitable, depending on the specific dimensions of the wound.

The invention will be described in greater detail, by way of example only, with reference to the accompanying drawings, in which

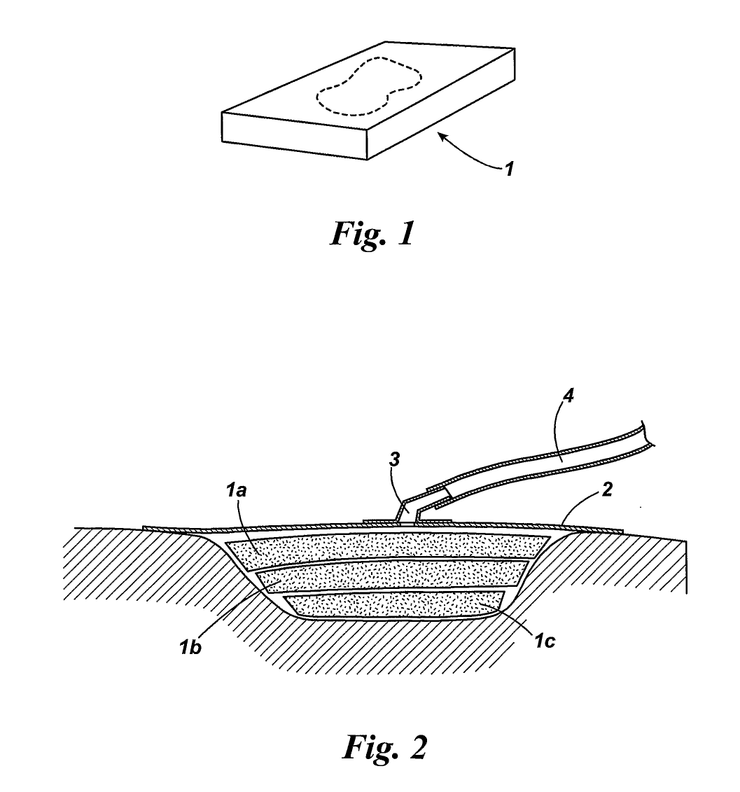

FIG. 1 is a perspective view of a sheet of a wound packing material according to the invention, with an indication of how the material may be cut to the appropriate dimensions to fit a wound cavity; and

FIG. 2 is a cross-sectional view, schematic and not to scale, of a wound cavity packed with a layered assembly of wound packing elements cut from the sheet of the type shown in FIG. 1, and with an occlusive wound dressing including a port by which negative pressure may be applied to the wound.

Referring first to FIG. 1, a sheet of wound packing material according to the invention is generally designated 1. On the surface of the sheet 1 is indicated (by a broken line) the outline of a part of the sheet 1 with a shape and dimensions to match a wound cavity. That part can be cut out to provide a wound packing element of the suitable dimensions for packing the wound cavity.

Referring now to FIG. 2, a wound cavity is packed with three wound packing elements 1a,1b,1c. The wound packing elements 1a,1b,1c are each cut from a sheet as shown in FIG. 1 and are of progressively decreasing dimensions, so that they form a layered assembly that fits closely within the wound cavity. In an alternative arrangement, wound packing elements may be supplied in a range of preformed sizes, from which suitable wound packing elements for the assembly of an appropriately-sized stack are selected. In negative pressure wound therapy it is desirable for correct functionality that the wound packing is of the correct size so that it is in contact with substantially the entire surface of the wound cavity before negative pressure is applied.

As shown in FIG. 2, the assembly of wound packing elements 1a,1b,1c is covered by an occlusive wound dressing consisting of a drape 2 and a drainage port 3, which may be connected via a tube 4 to a negative pressure source (not shown). The application of the negative pressure results in the contraction of the wound cavity, along with the enclosed wound packing. The presence of the wound packing prevents the dressing being drawn into the wound by suction, and ensures an even distribution of pressure throughout the wound cavity. Wound exudate is drawn from the wound, through the wound packing material and is removed via the tube 4.

1-24. (canceled)

25. A method of negative pressure wound therapy, which method comprises packing a wound cavity with one or more wound packing elements, and applying reduced pressure to the wound cavity, wherein the wound packing element is in the form of an airlaid nonwoven material.

26. A method as claimed in claim 25, wherein the wound packing element has the form of a planar sheet.

27. A method as claimed in claim 26, wherein the sheet has a thickness of from about 5 mm to several tens of millimetres.

28. A method as claimed in claim 26, wherein the thickness of the wound packing element is greater than 5 mm.

29. A method as claimed in claim 28, wherein the thickness of the wound packing element is greater than 10 mm.

30. A method as claimed in claim 26, wherein the wound packing element has a thickness that is less than 50 mm.

31. A method as claimed in claim 6, wherein the wound packing element has a thickness of less than 40 mm.

32. A method as claimed in claim 31, wherein the wound packing element has a thickness of less than 30 mm.

33. A method as claimed in claim 26, wherein the thickness of the wound packing element is in the range 5 mm to 50 mm.

34. A method as claimed in claim 33, wherein the thickness of the wound packing element is in the range 10 mm to 50 mm.

35. A method as claimed in claim 25, wherein the wound packing element is manufactured in a standard size and shape.

36. A method as claimed in claim 25, wherein the wound packing element is supplied in the form of an oversized sheet which may be cut to the appropriate dimensions immediately prior to use, to fit a wound cavity.

37. A method as claimed in claim 25, wherein the airlaid nonwoven material is thermally bonded.

38. A method as claimed in claim 25, wherein the airlaid nonwoven material is made from synthetic polymeric material.

39. A method as claimed in claim 38, wherein the synthetic polymeric material is polyester.

40. A method as claimed in claim 25, wherein the wound packing element is coated with a porous, non-adherent layer.

41. A method as claimed in claim 25, wherein the airlaid nonwoven material is coated or impregnated with one or more agents that complement its function as a wound packing element.

42. A method as claimed in claim 41, wherein the one or more agents are selected from agents to enhance the healing process, painkillers and antimicrobials.

43. A method as claimed in claim 25, wherein the wound packing element is pre-sterilised and supplied in sealed packaging.

44. A method as claimed in claim 25, wherein two or more wound packing elements are formed into a layered assembly, and positioned within the wound cavity.

45. A method of packing a wound cavity, which method comprises the insertion into the wound cavity of one or more wound packing elements, wherein the wound packing elements are in the form of an airlaid nonwoven material.

46. A wound packing comprising a layered assembly of two or more wound packing elements, wherein the wound packing elements are in the form of an airlaid nonwoven material.
